(19) United States
(12) Patent Application Publication
(10) Pub. No.: US 2018/0061221 A1
(43) Pub. Date: Mar. 1, 2018

(54) WEARABLE DEVICES

(71) Applicant: BOE TECHNOLOGY GROUP CO., LTD., Beijing (CN)

(72) Inventors: Yuedi He, Beijing (CN); Long Wang, Beijing (CN)

(21) Appl. No.: 15/647,236

(22) Filed: Jul. 11, 2017

(30) Foreign Application Priority Data
Aug. 29, 2016 (CN) 201620963172.3

(51) Int. Cl. G08C 17/02; A61B 5/02; A61B 5/00; A61B 5/024

(52) U.S. Cl. CPC G08C 17/02; A61B 5/02; A61B 5/0015; A61B 2562/0219; A61B 5/02438; A61B 5/68; A61B 5/45; A61B 5/00

(57) ABSTRACT

A wearable device comprises a detection unit configured to detect characteristic data of a user, wherein the characteristic data indicates an environment, a state and/or an action related to the user; a processing unit connected to the detection unit and configured to process the characteristic data detected by the detection unit to generate a trigger signal; and a display unit connected to the processing unit and configured to display a visual signal associated with the characteristic data according to the trigger signal generated by the processing unit.

### CROSS-REFERENCE TO RELATED APPLICATION(S)

This application claims priority to the Chinese Patent Application No. 201620963172.3, filed on Aug. 29, 2016, entitled “WEARABLE DEVICES,” which is incorporated herein by reference in its entirety.

### TECHNICAL FIELD

The present application relates to the field of communication technology, and more particular, to wearable devices.

### BACKGROUND

In today's bustling urban life, people often engage in social activities. Therefore, people need to dress up generously, decently and beautifully not only to give others a different feeling but also to give themselves enjoyment of beauty. This is particularly concerned by women. People may choose to wear different clothes and corresponding accessories (such as a necklace, an earring, a ring, a bracelet, a brooch etc.) on various occasions, which can reflect different styles, to bring different visual enjoyment to others. Although some accessories are exquisite and some other accessories are noble and generous, accessories which are currently available on the market mostly have a single function, that is, these accessories only have beautiful appearance, but cannot achieve intelligent applications. On the other hand, as requirements for personal data processing and communication continuously emerge, users want to acquire portable and personalized services whenever and wherever possible. Thus, it is desirable to integrate a portable device into a cloth or accessory of a user and achieve applications which intelligently vary by sensing different requirements.

### SUMMARY

According to a first aspect of the embodiments of the present application, there is provided a wearable device, comprising: a detection unit configured to detect characteristic data of a user, wherein the characteristic data indicates an environment, a state and/or an action related to the user; a processing unit connected to the detection unit and configured to process the characteristic data detected by the detection unit to generate a trigger signal; and a display unit connected to the processing unit and configured to display a visual signal associated with the characteristic data according to the trigger signal generated by the processing unit.

According to an embodiment of the present application, the characteristic data may comprise at least one of audio data, brightness data, temperature data, humidity data, force data and physiological data.

According to an embodiment of the present application, the audio data may indicate at least one of a volume, a tone, a speech rate and a voice command.

According to an embodiment of the present application, the visual signal may comprise optical signals having different colors, brightness and/or flicker frequencies.

According to an embodiment of the present application, the detection unit may comprise at least one of a sound sensor, a brightness sensor, a temperature sensor, a humidity sensor, a pressure sensor and a PhotoPlethysmoGraphy sensor.

According to an embodiment of the present application, the wearable device may further comprise: a communication unit connected to the processing unit and configured to communicate with other devices according to the trigger signal generated by the processing unit.

According to an embodiment of the present application, the other devices may comprise a mobile communication device capable of short-distance communication with the wearable device.

According to an embodiment of the present application, the trigger signal comprises an alarm trigger signal generated in response to the characteristic data indicating that the user wants to raise an alarm, wherein the alarm trigger signal triggers the communication unit to communicate with the mobile communication device to cause the mobile communication device to raise an alarm signal.

According to an embodiment of the present application, the mobile communication device raising the alarm signal may comprise: the mobile communication device automatically dialing a preset number and/or transmitting a message to the preset number.

According to an embodiment of the present application, the wearable device may access to a cloud service center through communication with the other devices via the communication unit.

According to an embodiment of the present application, the characteristic data of the user detected by the detection unit may be stored in the cloud service center through the communication with the other devices.

According to an embodiment of the present application, the wearable device may further comprise: a control unit connected to the detection unit and/or the processing unit and configured to control at least one of the following operations: detecting, by the detection unit, a specified kind of characteristic data, and generating, by the processing unit, the trigger signal.

### BRIEF DESCRIPTION OF THE DRAWINGS

In order to more clearly illustrate the technical solutions according to the embodiments of the present application, the accompanying drawings of the exemplary embodiments will be briefly described below. It will be apparent that the accompanying drawings in the following description are exemplary and illustrative only and are not intended to limit the present application in any way. Other accompanying drawings will be apparent to those of ordinary skill in the art from these accompanying drawings. Various aspects and their further purposes and advantages of the embodiments of the present application will be better understood with reference to the following detailed description of illustrative embodiments when read in conjunction with the accompanying drawings in which:

FIG. 1 is a structural diagram of a wearable device according to at least one embodiment of the present application;

FIG. 2 is a diagram of an intelligent earring according to at least one embodiment of the present application;

FIG. 3 is a structural diagram of a wearable device according to at least one embodiment of the present application;

FIG. 4 is a diagram of another intelligent earring according to at least one embodiment of the present application;

FIG. 5 is a structural diagram of a wearable device according to at least one embodiment of the present application;

FIG. 6 is a diagram of an intelligent earring having a switch apparatus according to at least one embodiment of the present application; and

FIG. 7 is a diagram of a switch apparatus according to at least one embodiment of the present application.

### DETAILED DESCRIPTION

In order to make the purposes, technical solutions and advantages of the embodiments of the present application more clear, the embodiments of the present application will be described in detail below with reference to the accompanying drawings. Obviously, the described embodiments are only a part of the embodiments of the present application, instead of all the embodiments. All other embodiments obtained by those of ordinary skill in the art based on the embodiments of the present application without contributing any creative labor are within the protection scope of the present application.

Throughout this specification, reference to features, advantages, or similar expressions is not intended to mean that all features and advantages which may be practiced with the present application should be or are within any single embodiment of the present application. Rather, it is to be understood that expressions related to features and advantages mean that the specific features, advantages or characteristics described in connection with the embodiments are included in at least one embodiment of the present application. Thus, throughout the specification, discussions of features and advantages as well as similar expressions may refer to the same embodiment, but not necessarily to the same embodiment. In addition, the described features, advantages and characteristics of the present application may be incorporated in one or more embodiments in any suitable manner. Those skilled in the relevant art will recognize that the present application may be practiced without one or more specific features or advantages of a particular embodiment. In other examples, additional features and advantages may be implemented in certain embodiments, but are not necessarily present in all the embodiments of the present application.

The embodiments of the present application provide a wearable device which can be variously applied with demands in different scenarios, so as to provide personalized services for personal data processing and communication.

FIG. 1 is a structural diagram of a wearable device according to at least one embodiment of the present application. As shown in FIG. 1, the wearable device 100 according to the embodiment of the present application may comprise a detection unit 101, a processing unit 102 connected to the detection unit 101, and a display unit 103 connected to the processing unit 102. According to the embodiment of the present application, the detection unit 101 may be used to detect characteristic data of a user, wherein the characteristic data indicates an environment, a state and/or an action related to the user. The processing unit 102 may be used to process the characteristic data detected by the detection unit 101 to generate a trigger signal. The display unit 103 may be used to display a visual signal associated with the characteristic data according to the trigger signal generated by the processing unit 102.

It is to be noted that terms such as “connected with”, “connected to” etc. referred to in describing the embodiments of the present application may refer to direct or indirect coupling between various units in a device and/or between the device and another device outside the device, which may be achieved in a wired or wireless manner.

According to an embodiment of the present application, the detection unit 101 may comprise at least one of a sound sensor, a brightness sensor, a temperature sensor, a humidity sensor, a pressure sensor and a PhotoPlethysmoGraphy (PPG) sensor. For example, one or more sensor technology may be used in the wearable device 100, and the detection unit 101 and corresponding functions thereof may be implemented by a single independent sensor in a centralized manner or by a plurality of separate sensors in a distributed manner.

According to an embodiment of the present application, the characteristic data of the user detected by the detection unit 101 may comprise at least one of audio data, brightness data, temperature data, humidity data, force data and physiological data. For example, the characteristic data may provide information related to an environment in which the user of the wearable device 100 is located, such as information indicating a temperature, humidity, light brightness and/or a background sound etc. of the user environment. In addition, the characteristic data may provide information related to a state of the user of the wearable device 100, such as information indicating a cardiac function, a blood flow and/or emotional fluctuation etc. of the user. Further, the characteristic data may further provide information related to an action of the user of the wearable device 100, such as information indicating that the user has pressed and/or touched at least a portion of the wearable device 100 etc.

According to an embodiment of the present application, the audio data may indicate at least one of a volume, a tone, a speech rate, and a voice command. For example, the audio data may reflect a change in volume, tone and speech rate, and/or is associated with particular context and semantics when the user of the wearable device 100 speaks. It will be appreciated that the audio data detected by the detection unit 101 may comprise sound information from the user himself/herself of the wearable device 100 and/or sound information from other people or objects surrounding the user.

The processing unit 102 of the wearable device 100 may generate a trigger signal by processing the characteristic data detected by the detection unit 101 so as to trigger the display unit 103 to display a visual signal associated with the characteristic data, thereby reflecting the environment, state and/or action related to the user in a visual manner. According to an embodiment of the present application, the visual signal may comprise optical signals having different colors, brightness and/or flicker frequencies.

According to an embodiment of the present application, specific values or ranges of values of various kinds of characteristic data may be associated with visual signals having different display characteristics. For example, an optical signal with a cool tone is displayed in a high temperature environment, an optical signal with a warm tone is displayed in a dark environment, an optical signal with high brightness is displayed when the user is talking loudly, an optical signal with low brightness is displayed when a pulse of the user is steady, an optical signal with a predetermined flicker frequency is displayed when the user touches the wearable device, a specified optical signal is displayed according to a voice command issued by the user etc. Thus, the wearable device 100 according to the embodiment of the present application may have variable appearance display with the environment and/or according to requirements of the user, so as to adapt to different application occasions.

On the other hand, in addition to reflecting different circumstances related to the user through the appearance display, the wearable device 100 according to the embodiment of the present application can further realize monitoring and management of user personal data such as health indexes etc. by detecting and collecting the characteristic data. In order to detect and collect physiological data related to health conditions and/or physical conditions of the user, various biological and medical detection technology and means, such as PhotoPlethysmoGraphy (PPG) and Heart Rate Variability (HRV) may also be used.

PPG is a noninvasive method for detecting a change in a blood volume in living tissues by optoelectronic means. When a light beam at a certain wavelength is illuminated onto a surface of a skin, the light beam may be transmitted or reflected to a photoelectric receiver. In this process, light intensity detected by a detector may decrease under the action of absorption and attenuation by muscles and blood of the skin. In particular, absorption of light by muscular tissues etc. of the skin maintains constant throughout the blood circulation, while the blood volume within the skin changes in a pulsatile manner under the action of the heart. When the heart is contracted, a peripheral blood volume is the largest, a light absorption amount is also the largest, and the detected light intensity is the smallest; and on the contrary, during diastole of the heart, the detected light intensity is the largest, so that the light intensity received by the light receiver changes in a pulsatile manner therewith. A change signal of the light intensity is converted into an electrical signal, and then a change of a volume pulse blood flow may be acquired. Thus, the volume pulse blood flow contains important physiological information of the cardiovascular system such as a heartbeat function, a blood flow etc. At the same time, the volume pulse blood flow mainly exists in micro-vessels such as micro-arteries, capillaries in peripheral blood vessels, and therefore the volume pulse blood flow also contains a variety of physiological and pathological microcirculation information, which is an important information source for research on the circulatory system of human bodies.

In this regard, the detection unit 101 of the wearable device 100 according to the embodiment of the present application can be implemented as a PPG sensor using the PPG technology, which can collect the physiological data and/or pathological data of the user. In this way, the characteristic data detected by the detection unit 101 may comprise not only information which reflects an external environment of the user, but may also comprise information which reflects an inner physiological condition of the user. Thus, for users who are increasingly concerned with physical and physiological health, the wearable device 100 according to the embodiment of the present application can not only serve as an important accessory with decorative and aesthetic effects, but also can serve as an intelligent wearable product for detecting health indexes, for example, in conjunction with the Internet of Things technology.

The wearable device according to the embodiment of the present application may be implemented as or integrated into various accessories such as an earring, a necklace, a watch, a ring, a bracelet, a waistband, a broom, a hair card, eyeglasses etc. As another example, the wearable device according to the embodiment of the present application may also be implemented as a detachable component of various accessories such as a pendant of a necklace or earring, a wrist strap of a watch or bracelet, a buckle of a waistband or belt, an inlay of a ring or brooch etc. In yet another example, various structural units of the wearable device according to the embodiment of the present application may also be implemented in one or more accessories or in different components of the same accessory in a distributed manner.

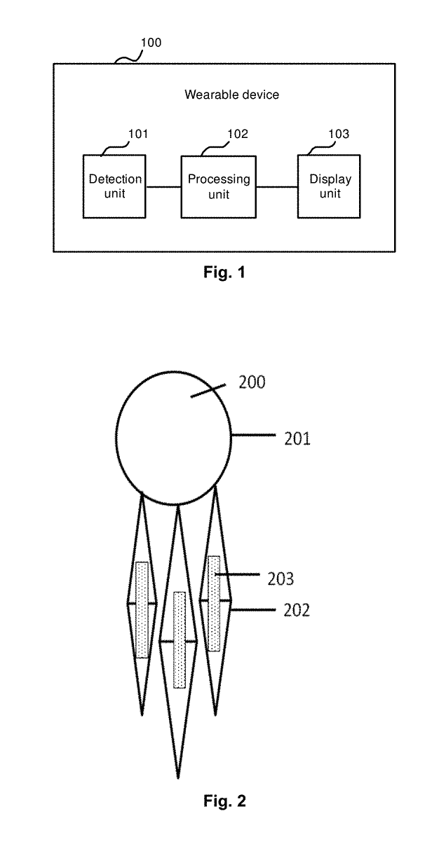

FIG. 2 is a diagram of an intelligent earring according to at least one embodiment of the present application. The wearable device 100 as described in connection with FIG. 1 may be implemented as an intelligent earring 200 as shown in FIG. 2, wherein the intelligent earring 200 comprises an ear stud 201 and a pendant 202. For example, one or more units in the wearable devices 100 shown in FIG. 1 may be provided on the ear stud 201 and remaining units (if any) may be provided on the pendant 202. It is to be noted that due to a wide variety of earrings (for example, a nail earring, a ring-shaped earring, a hanging earring, a clip-like magnetic earring, a silicone earring, etc.), the intelligent earring according to the embodiment of the present application does not necessarily comprise a pendant, and instead may only comprise an ear stud, or the pendant may be implemented as a detachable part of the earring.

In the example shown in FIG. 2, a detection unit and a processing unit of the wearable device according to the embodiment of the present application are provided on the ear stud 201, and a display unit 203 of the wearable device according to the embodiment of the present application is provided on the pendant 202. For example, the display unit 203 may comprise a color-variable fluorescent lamp or a light emitting apparatus made of other color-variable materials, such as a photochromic material. Accordingly, the pendant 202 may be made of a transparent color material, which enables brightness of the visual signal of the display unit 203, such as a fluorescent lamp, to diverge.

According to an embodiment of the present application, the detection unit (for example a sound sensor) of the intelligent earring 200 may detect characteristic data of a user or in a surrounding environment of the user, such as audio data indicating a volume when the user speaks etc., and the processing unit of the intelligent earring 200 may generate a trigger signal (for example, for triggering the display unit 203 to display a visual signal corresponding to the detected volume) according to the detected characteristic data, so that different visual signals may be displayed by the display unit 203 to cause the intelligent earring 200 to change an appearance thereof with the environment (for example, changing brightness and/or color of the fluorescent lamp on the pendant with the volume).

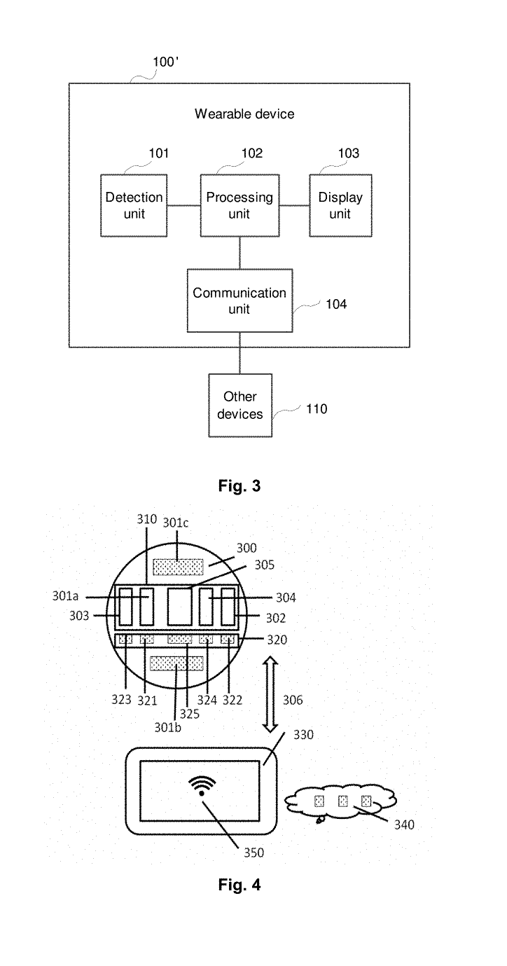

FIG. 3 is a structural diagram of a wearable device according to at least one embodiment of the present application. As shown in FIG. 3, in addition to the detection unit 101, the processing unit 102 and the display unit 103, a wearable apparatus 100′ according to the embodiment of the present application may comprise a communication unit 104 connected to the processing unit 102 and configured to communicate with other devices 110 according to a trigger signal generated by the processing unit 102. For example, the communication may comprise wired and/or wireless communications in accordance with various communication protocols and specifications. Accordingly, the other devices 110 may comprise stationary and/or mobile terminals in accordance with various communication standards and specifications, including, but not limited to, a mobile phone, a smart phone, a multimedia device, a desktop computer, a laptop computer, a tablet computer, a personal communication system device, a personal navigation device, a personal digital assistant, a digital camera, a digital video camera, a positioning device, and/or a medical device etc.

According to an embodiment of the present application, the other devices 110 may comprise a mobile communication device capable of short-distance communication with the wearable device 100. For example, the short-distance communication may be supported by a wireless communication medium such as Bluetooth, infrared, Near Field Communication (NFC), Wireless Local Area Network (WLAN) etc., or may be supported by a wired connection manner such as Universal Serial Bus (USB)/fire wire connection etc.

As described in connection with FIG. 1, the processing unit 102 may generate a trigger signal to trigger the display unit 103 to display an associated visual signal. In addition, the processing unit 102 may also generate various trigger signals for triggering other structural units of the wearable device 100 to perform corresponding operations. According to an embodiment of the present application, the trigger signal generated by the processing unit 102 of the wearable device 100 may comprise an alarm trigger signal generated in response to the detected characteristic data indicating that the user wants to raise an alarm. The alarm trigger signal triggers the communication unit 104 of the wearable device 100 to communicate with the mobile communication device, so as to cause the mobile communication device to raise an alarm signal. According to an embodiment of the present application, the mobile communication device raising the alarm signal may comprise: the mobile communication device automatically dialing a preset number and/or transmitting a message to the preset number.

At present, the safety problem of women has always been a hot topic of social concern. When a female user encounters danger, it is very important for the protection of personal property security of the female user to call the police in a case that criminals are not easy to discover the action of the female user. For example, the female user may wear a wearable device (for example, an earring) according to an embodiment of the present application, and press and/or touch at least a portion of the wearable device (for example, a pressure sensor or a switch apparatus) in a critical situation, to cause the wearable device to communicate and interconnect with a mobile phone of the female user, thereby controlling the mobile phone to dial the police emergency number or transmit information. This manner of calling the police is simple and convenient, has imperceptibility, and is not easy to be discovered by criminals who are committing crimes, which can effectively protect women.

With the rapid development of modern science and technology, the Internet has rapidly evolved into the mobile Internet era including the Internet of Things, cloud computing and large data. As described above, the wearable device 100 according to the embodiment of the present application may communicate with other devices 110 (for example, stationary and/or mobile terminals) through the communication unit 104. In particular, the wearable device may access to a cloud service center through communication between the communication unit 104 and the other devices 110. For example, the cloud service center may comprise an intelligent large data cloud center associated with a specific service and the cloud service center is accessible via a mobile communication network and/or the Internet etc., so as to implement data exchange with the cloud service center.

According to an embodiment of the present application, the characteristic data of the user detected by the detection unit 101 of the wearable device 100 may be stored in the cloud service center via communication with the other devices 110. For example, the processing unit 102 of the wearable device 100 may perform processing such as separation, conversion and/or extraction etc. on the detected characteristic data, provide at least a portion of the processed characteristic data to the communication unit 104, and trigger the communication unit 104 to perform data communication with at least one other device which may access to the cloud service center by generating a communication trigger signal, so as to implement cloud storage of the characteristic data.

According to an embodiment of the present application, the cloud service center may monitor the characteristic data of the user in real time and realize interaction with the user. For example, a functional module (which, for example, performs a specified application) associated with the cloud service center may be enabled on other devices (for example, smart phone) which communicates with the wearable device 100, and the cloud service center may establish a human-computer interaction link with the user through this functional module. On this basis, the cloud service center may provide feedback information (such as storage logs, health reports, etc.) to the user by monitoring the characteristic data of the user in real time, and acquire input information (for example parameter settings, personal profiles, etc.) from the user, so as to achieve human-computer interaction.

In an exemplary wearable device according to an embodiment of the present application, the detection unit may monitor a pulse change of a wearer of the wearable device in real time, and the processing unit may perform feedback (for example, cause a change in appearance display or otherwise transmit prompt information) according to the detected data, so that the wearer maintains a stable mood as much as possible. On the other hand, the wearable device may separate pulse information detected by the detection unit in real time through the processing unit and/or other functional unit, and store the pulse information in the cloud service center through communication with the other devices 110 (for example, a smart phone) via the communication unit 104, so as to achieve cloud storage of diary records of personal pulse information, and thus provide strong data support of long-term tracking of the pulse information.

FIG. 4 is a diagram of another intelligent earring according to at least one embodiment of the present application. The wearable device 100′ as described in connection with FIG. 3 may be implemented as an intelligent earring 300 as shown in FIG. 4. Specifically, the detection unit 101 of the wearable device 100′ may be provided as sensors 301a, 301b and 301c in the intelligent earring 300, the processing unit 102 may be provided as a microprocessor 302, the display unit 103 may be provided as a light emitting body 303, and the communication unit 104 may be provided as an I/0 transceiver 304. Alternatively or additionally, the intelligent earring 300 as shown in FIG. 4 may further comprise a battery 305 for supplying power. It will be appreciated that the intelligent earring 300 may further comprise a removable pendant, and at least one component (for example, the display unit 103) in the intelligent earring 300 may be placed on the pendant as appropriate.

For example, the sensor 301a may comprise a PPG sensor capable of reflecting HRV state parameters, the sensor 301b may comprise a sound sensor, the sensor 301c may comprise a pressure sensor acting as an alarm sensor, the microprocessor 302 may comprise a low power microprocessor, the light emitting body 303 may comprise an LED light (which may also provide a light source for the PPG sensor), and the I/O transceiver 304 may comprise an I/O Bluetooth 4.0 transceiver for wireless communication. With the PPG and HRV technology, Internet of things and large data technology and a variety of sensor technology, the intelligent earrings 300 as shown in FIG. 4 can not only monitor health conditions, but also comprise an alarm system and can change a color thereof with the environment.

The intelligent earring 300 according to the embodiment of the present application integrates a system design which enables adaptive change of appearance display, control of alarms, monitoring of user characteristic data etc., and in a context of the Internet of Things, the system design may comprise a chip layer 310, a carrier board layer 320, a gateway layer 330, a central layer 340, a presentation layer 350, and corresponding inter-layer protocol designs thereof. For example, the intelligent earring 300 may have a special metal bracket with a certain resilience embedded therein as a carrier for the system design.

As shown in FIG. 4, components of the sensor 301a, the microprocessor 302, the light emitting body 303, the I/O transceiver 304 and the battery 305 may be provided on the chip layer 310, and a sensor component carrier board 321, a microprocessor component carrier board 322, a light emitting body component carrier board 323, an I/O transceiver component carrier board 324 and a battery component carrier board 325 may be correspondingly provided on the carrier board layer 320. Similarly, components of the sensors 301b and 301c and carrier boards thereof may be provided on the chip layer 310 and the carrier board layer 320 respectively. A circuit may be loaded for each chip component carrier board on the chip layer 310 according to a structure and a shape of the earring accessory and a preconfigured design may be realized in accordance with interconnection setting of each carrier board.

According to an exemplary embodiment, a carrier of the gateway layer 330 may be a smart phone connectable to the Internet. Accordingly, the intelligent earring 300 may access to a Bluetooth wireless communication link 306 through Bluetooth communication with the smart phone (for example, via the microprocessor 302 and the I/O transceiver 304), and then access to an intelligent cloud center of the Internet via the Bluetooth interface (which may act as an interface to an access gateway of the cloud center of the Internet) of the smart phone and corresponding gateway conversion.

According to an exemplary embodiment, a carrier of the central layer 340 may be the cloud service center of the Internet, which may provide services such as large data analysis and monitoring, cloud storage of user characteristic data etc. In order to more intuitively provide and present services of the intelligent cloud center to the user, monitoring of the user characteristic data may be set by the presentation layer 350. For example, design of the presentation layer 350 may be implemented by running an App application which pushes the monitoring service on the smart phone.

Thus, the intelligent earring 300 as shown in FIG. 4 may access to the cloud center through connection to the Internet, so as to achieve real-time data monitoring under the control of the cloud center. On the other hand, as the intelligent earring 300 interconnected with the smart phone is also equipped with an alarm function, the user may control the smart phone to raise an alarm signal in a critical situation. Further, the intelligent earring 300 has a function of adaptively displaying a visual signal, and therefore the intelligent earring 300 may change the appearance display (for example, changing a color of the pendant etc.) depending on an application scene.

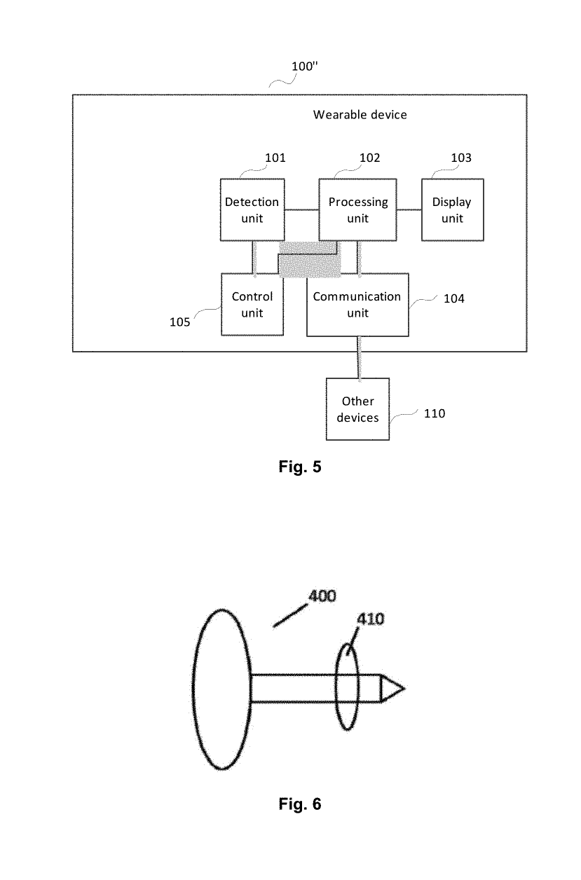

FIG. 5 is a structural diagram of a wearable device according to at least one embodiment of the present application. As shown in FIG. 5, in addition to the detection unit 101, the processing unit 102, the display unit 103 and the communication unit 104, the wearable device 100″ according to the embodiment of the present application may further comprise a control unit 105 connected to the detection unit 101 and/or the processing unit 102. The control unit 105 may be used to control at least one of the following operations: detecting, by the detection unit 101, a specified kind of characteristic data, and generating, by the processing unit 102, a trigger signal.

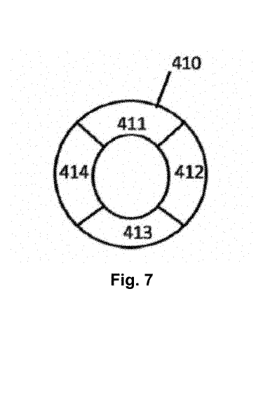

For example, the control unit 105 may be implemented as a functional button or a switch apparatus (as described below in connection with FIG. 6). By manipulating the switch or pressing the button, the user may control the detection unit to detect specific characteristic data only, for example, the user may selectively enable one or more related sensors to detect the characteristic data. Alternatively or additionally, by manipulating the switch or pressing the button, the user may also control whether the processing unit 102 generates a specific trigger signal, for example, the user may select to disable the communication unit 104 and accordingly not to generate a communication trigger signal.

Thus, by appropriately operating the control unit 105, it is possible to select and switch among various functions of the wearable device according to the embodiment of the present application. For example, an alarm function may be integrated within the wearable device, and when a female user encounters a critical situation, it is possible to automatically control a mobile phone interconnected to the wearable device to call the police by touching at least a portion of the wearable device (for example, turning on the switch or pressing a specific button).

Alternatively or additionally, the user may select to use the wearable device as an accessory which may change a color thereof with the environment (for example, an earring equipped with a removable luminous pendant), so that the user can not only wear the accessory as a regular ornament, but also can change the color of accessory in different circumstances (for example, when volumes are different), thereby giving romantic and changeable feelings to people. Further, the accessory is easy to implement, and can adapt to requirements on different occasions.

Alternatively or additionally, the user may also select to use the wearable device as a health condition monitoring product, and by using the Internet of Things sensing technology, cloud computing and large data technology, the product can achieve cloud storage of diary records of personal pulse information and provide strong data support for long-term tracking of the pulse information in a case of mobile pulse detection.

It is to be understood that the user may select to simultaneously enable a plurality of functions of the wearable device, thereby enabling the wearable device, for example, not only to change the appearance display with the environment, but also to be connected to a variety of wired and/or wireless networks via communication with other devices so as to obtain different network services (for example cloud storage, etc.).

FIG. 6 is a diagram of an intelligent earring having a switch apparatus according to at least one embodiment of the present application. FIG. 6 illustrates a longitudinal cross-sectional view of an intelligent earring 400, which may be considered as the intelligent earring 300 as shown in FIG. 4 having a switch apparatus 410. In the exemplary embodiment, the intelligent earring 400 is an intelligent earring having functions such as pulse detection, alarming, and color change with the environment. Accordingly, FIG. 7 is a diagram of a switch apparatus according to at least one embodiment of the present application. As shown in FIG. 7, the switch apparatus 410 has a function close button 411, a switch button 412 for controlling a pulse monitoring system, a switch button 413 for controlling an alarm system, and a switch button 414 for controlling a color-variable fluorescent lamp system. When the function close button 411 is pressed down, functions of the intelligent earring 400 may be disabled.

According to an exemplary embodiment, when the switch button 412 is pressed down, the intelligent earring 400 is operable as an Internet of Things intelligent earring. For example, the intelligent earring 400 may cause its embedded microprocessor and its connected PPG sensor, Bluetooth wireless transceiver, etc. to be in a normal operating state. In a case that the smart phone acting as an Internet access gateway is also in a normal operating state, the intelligent earring 400 may be connected to a gateway layer through corresponding devices on a chip layer and a carrier board layer to access to a central layer in the Internet, thereby accessing to an intelligent large data cloud center of the intelligent earphone 400.

When a user wears the intelligent earring 400, its embedded microprocessor may perform real-time processing at a specific period (for example, every 10 minutes) continuously (for example, for 24 hours), to convert for example HRV data parameters which are collected and calculated via the PPG sensor into pulse state data parameters, and transfer these data parameters to the intelligent large data cloud service center via the smart phone through the Internet, for storage as a log file. In addition, the intelligent large data cloud service center may implement a human-computer interaction link from the user to the cloud service center and perform real-time monitoring of a pulse state of the user locally and in the cloud center by running a corresponding App application on the smart phone.

When an emergency situation is encountered and thus an alarm is required to be raised, the user may press the alarm switch button 413 down, so that the embedded alarm sensor is in a normal operating state, and at the same time, the intelligent earring 400 may be triggered to be connected to the smart phone and start a corresponding application in response to a signal from the alarm sensor, so as to make a call or transmit information to a set number.

When the user wishes the intelligent earring 400 to be able to change display of a fluorescent lamp with different occasions or in accordance with an instruction from the user, the switch button 414 may be pressed down by the user, so that the embedded sound sensor and the color-variable fluorescent lamp are in a normal operating state. The sound sensor is used to detect audio information (for example, a volume when the user speaks etc.), so that the fluorescent lamp may change its brightness and/or color etc. in accordance with an indication of the audio information.

It is to be noted that although the intelligent earring 400 shown in FIG. 6 changes appearance display according to the audio information, those skilled in the art will recognize that the wearable device according to the embodiments of the present application can also trigger different displays of the visual signal according to other characteristic data. Similarly, although the intelligent earring 400 shown in FIG. 6 monitors the pulse state of the user, those skilled in the art will recognize that the wearable device according to embodiments of the present application can also utilize other detection units to collect and monitor other types of user characteristics data.

It is to be noted that FIGS. 2 to 7 schematically illustrate only the wearable devices implemented in an intelligent earring manner, and one of ordinary skill in the art can contemplate other means for implementing the wearable device according to the embodiments of the present application on the basis of the structures shown in FIGS. 1-7 without contributing any creative labor. For example, the wearable device according to the embodiments of the present application can further be implemented as any other suitable intelligent accessory such as a necklace, a watch, a ring, a bracelet, a waistband, a brooch, a hair card, glasses etc.

It should be understood that FIGS. 1 to 7 schematically illustrate only exemplary structures of the wearable devices according to embodiments of the present application, and in practical production and applications, one of ordinary skill in the art can contemplate various equivalent structures or variations of the wearable device according to the embodiments of the present application based on the teachings provided in this specification. For example, the wearable device may have more or fewer units and/or devices, and the wearable device may be implemented using different types and/or modes of components as appropriate.

Many modifications and other embodiments of the present application set forth herein will be apparent to those of ordinary skill in the art to which the present application pertains from the benefit of the teachings presented in the foregoing description and associated accompanying drawings. Therefore, it is to be understood that the present application is not limited to the specific embodiments disclosed and is intended to cover such modifications and other embodiments within the scope of the appended claims.

What is claimed is:

1. A wearable device, comprising:
a detection unit configured to detect characteristic data of a user, wherein the characteristic data indicates an environment, a state and/or an action related to the user;
a processing unit connected to the detection unit and configured to process the characteristic data detected by the detection unit to generate a trigger signal; and
a display unit connected to the processing unit and configured to display a visual signal associated with the characteristic data according to the trigger signal generated by the processing unit.

2. The wearable device according to claim 1, wherein the characteristic data comprises at least one of audio data, brightness data, temperature data, humidity data, force data and physiological data.

3. The wearable device according to claim 2, wherein the audio data indicates at least one of a volume, a tone, a speech rate and a voice command.

4. The wearable device according to claim 1, wherein the visual signal comprises optical signals having different colors, brightness and/or flicker frequencies.

5. The wearable device according to claim 1, wherein the detection unit comprises at least one of a sound sensor, a brightness sensor, a temperature sensor, a humidity sensor, a pressure sensor and a PhotoPlethysmoGraphy sensor.

6. The wearable device according to claim 1, further comprising:
a communication unit connected to the processing unit and configured to communicate with other devices according to the trigger signal generated by the processing unit.

7. The wearable device according to claim 6, wherein the other devices comprise a mobile communication device capable of short-distance communication with the wearable device.

8. The wearable device according to claim 7, wherein the trigger signal comprises an alarm trigger signal generated in response to the characteristic data indicating that the user wants to raise an alarm, wherein the alarm trigger signal triggers the communication unit to communicate with the mobile communication device to cause the mobile communication device to raise an alarm signal.

9. The wearable device according to claim 8, wherein the mobile communication device raises the alarm signal by automatically dialing a preset number and/or transmitting a message to the preset number.

10. The wearable device according to claim 6, wherein the wearable device accesses to a cloud service center through communication with the other devices via the communication unit.

11. The wearable device according to claim 10, wherein the characteristic data of the user detected by the detection unit is stored in the cloud service center through the communication with the other devices.

12. The wearable device according to claim 1, further comprising:
a control unit connected to the detection unit and/or the processing unit and configured to control at least one of the following operations: detecting, by the detection unit, a specified kind of characteristic data, and generating, by the processing unit, the trigger signal.
